(19) United States
(12) Patent Application Publication
(10) Pub. No.: US 2025/0352818 A1
(43) Pub. Date: Nov. 20, 2025

(54) PHOTOTHERAPY APPARATUS

(71) Applicant: LUMITEX, INC., Strongsville, OH (US)

(72) Inventors: Michael KERNS, Strongsville, OH (US); Anton ZONNEVELD, Strongsville, OH (US); Joseph DOMBROWSKI, Strongsville, OH (US); Alan GRESZLER, Strongsville, OH (US)

(73) Assignee: LUMITEX, INC., Strongsville, OH (US)

(21) Appl. No.: 18/874,197

(22) PCT Filed: Jun. 23, 2023
(86) PCT No.: PCT/US2023/068959

§ 371 Date: Dec. 12, 2024

Related U.S. Application Data
(60) Provisional application No. 63/368,070, filed on Jul. 11, 2022.

(51) Int. Cl. A61N 5/06

(52) U.S. Cl. CPC A61N 5/0621; A61N 2005/0626; A61N 2005/0632; A61N 2005/0637; A61N 2005/0652

(57) ABSTRACT

A phototherapy apparatus is provided for delivering light to an infant. The phototherapy apparatus includes a light source sheet having light emitters that are electrically isolated from the external environment using an isolating covering. Light from the light emitters is diffused by a spacing layer before being emitted from the spacing layer.

This International Application claims priority to U.S. Provisional Application No. 63/368,070 filed Jul. 11, 2022, which is hereby incorporated herein by reference in its entirety.

### TECHNICAL FIELD

The present disclosure relates generally to a phototherapy apparatus and system for delivering light to an infant's torso.

### BACKGROUND

Phototherapy has long been used to treat newborn infants for various maladies including jaundice. Jaundice is caused by a buildup of bilirubin in the blood of infants. Exposing the infant's skin to certain types of light will quickly reduce the bilirubin to a safe level.

A common problem in the treatment of jaundice, especially in full-term babies, involves exposing a baby's skin to overhead lights (the light converting bilirubin to another molecule that kidneys typically can filter). Often when a baby is not tucked or swaddled, the baby panics, flails arms, and cries. This often causes a parent or nurse to end treatment prematurely, at the risk of jaundice levels remaining elevated.

### SUMMARY

The present disclosure provides a phototherapy apparatus for delivering light to an infant using an electrically isolated light source sheet and a spacing layer for diffusing the light emitted by light emitters of the light source sheet. An infant may be placed onto the phototherapy apparatus and the infant and the phototherapy apparatus may together may be wrapped in a covering.

While a number of features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the various ways in which the principles of the invention may be employed. Other objects, advantages, and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is a side view of an exemplary embodiment of a phototherapy apparatus.

FIG. 2 is a top view of the phototherapy apparatus of FIG. 1.

FIG. 3 is a side view of an exemplary embodiment of a phototherapy apparatus having a non-planar external surface.

FIG. 4 is a side view of an alternative exemplary embodiment of a phototherapy apparatus.

FIG. 5 is a top view of the phototherapy apparatus of FIG. 3.

FIG. 6 is a side view of an exemplary embodiment of a phototherapy apparatus illuminating an infant.

FIG. 7 is a top view of the phototherapy apparatus of FIG. 6.

FIG. 8 is a flow diagram of an exemplary method of manufacturing a phototherapy apparatus for delivering light to an infant.

### DETAILED DESCRIPTION

Jaundice is caused in most neonates and a certain percentage of full-term babies when the liver is inadequately developed to filter Bilirubin from the blood. Neonates and full-term babies with jaundice are commonly treated with blue light, which conjugates bilirubin molecules into lumirubin and photobilirubin, isomers of bilirubin which can be filtered by the kidneys.

The present disclosure provides a phototherapy apparatus for delivering light to an infant. The phototherapy apparatus includes a light source sheet having light emitters that are electrically isolated from the external environment using an isolating covering. Light from the light emitters is diffused within a spacing layer before being emitted from the spacing layer (e.g., to illuminate the infant).

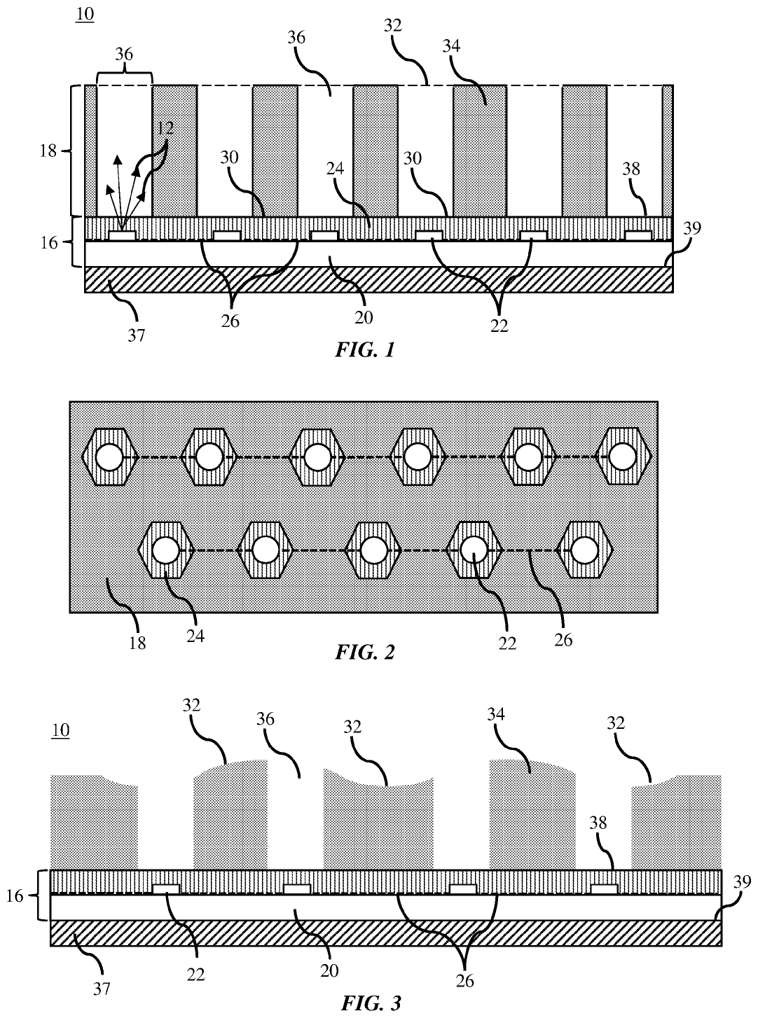

Turning to FIGS. 1 and 2, an exemplary embodiment is shown of a phototherapy apparatus 10 (also referred to as a phototherapy device) for delivering light 12 to an infant 14. The phototherapy apparatus 10 includes a light source sheet 16 and a spacing layer 18. The light source sheet 16 includes a backing 20, light emitters 22 for emitting light 12 that are mechanically supported by the backing 20, an isolating covering 24, and electrical pathways 26 located on the backing 20 for delivering electrical power to the light emitters 22. The isolating covering 24 electrically isolates the electrical pathways 26 and the light emitters 22 from an external environment 28. The spacing layer 18 diffuses the light 12 from the light emitters 22 before the light 12 is emitted from the spacing layer 18.

The spacing layer 18 may include an internal surface 30 and an external surface 32 opposite the internal surface 30. The spacing layer 18 may be made of a flexible material 34 including voids 36. The voids 36 may be positioned to overlap with the light emitters 22, such that the light 12 emitted by the light emitters 22 diffuses within the voids 36. A void 36 is referred to as associated with a light emitter 22 when the void 36 overlaps the light emitter 22 (e.g., the light 12 emitted by the light emitter 22 is transmitted onto the void 36).

As shown in FIG. 1, the internal surface 30 may be mechanically supported by the light source sheet 16. The voids 36 may extend between the internal surface 30 and the external surface 32. The voids 36 may take any suitable shape. For example, the voids 36 may be hexagonal cut outs, circular cut outs, etc.

As shown in FIG. 3, the external surface 32 may be non-planar. For example, the external surface 32 may include surface texturing (also referred to as contouring) including at least one of depressions or protrusions to improve comfort to the infant 12 and to improve air flow to skin of the infant 12. That is, the surface texturing may provide air pathways allowing heat and/or moisture to escape from between the infant's skin and the phototherapy apparatus 10.

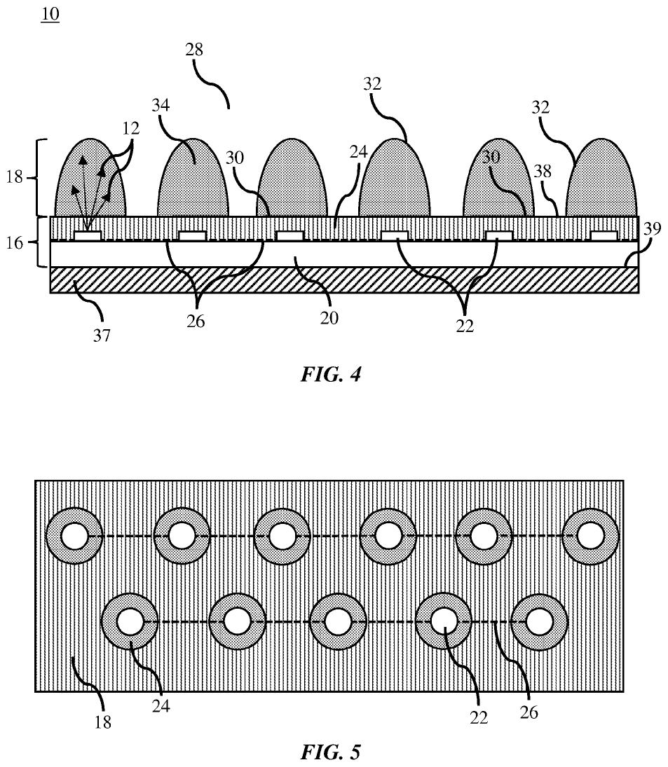

Turning to FIGS. 4 and 5, the spacing layer 18 may include lensing features 38 positioned to overlap with the light emitters 22. The lensing features 38 may be positioned such that the light 12 emitted by the light emitters 22 is received by the lensing features 38 and is emitted by the lensing features 38 having a desired light distribution (e.g., the light 12 may have a diffuse distribution upon being emitted from the lensing features 38). The lensing features 38 may be any suitable structure for diffusing light emitted from the light emitters 22. For example, the lensing features 38 may include a semi-spherical or elliptical optical element.

The spacing layer 18 may be made of any suitable material. For example, the spacing layer 18 may be made of a flexible material having sufficient rigidity for supporting an infant and for maintaining separation between the infant 14 and the light source sheet 16. As an example, the spacing layer 18 may include a closed cell foam (such as urethane, PET, PE) and/or an open cell foam that allows for air flow and moisture exchange. An outer surface of the spacing layer 18 may also act as a reflector to redirect electromagnetic radiation reflected by a skin surface (i.e., redirecting this reflected light back towards the skin surface).

The spacing layer 18 may also be affixed (e.g., adhered) to the light source sheet. For example, the spacing layer 18 may be adhered to the light source sheet 16 by overmolding the spacing layer 18 onto the light source sheet 16. As an example, the spacing layer 18 may be formed from molded silicone or urethane. Hydrogels may be added to the spacing layer 18 to improve oxygen permeability at the light emitting surface 38. The light source sheet 16 and the spacing layer 18 may be flexible and configured to conform to a skin surface of the infant 14.

In one embodiment, the spacing layer 18 may be a hydrogel that is at least partially transparent to the light emitted by the light emitters 22. The hydrogel may be configured to be biocompatible, such that the hydrogel may make direct contact with an infant's skin for a duration of time (e.g., multiple hours) without damaging the skin. For example, the spacing layer 18 may be formed from any gel such as polyurethane or any suitable material capable of wrapping around an infant.

The spacing layer 18 may have any thickness suitable for diffusion of the light 12 emitted by the light emitter 22. For example, the spacing layer 18 may have a thickness of at least 5 mm, at least 10 mm, at least 20 mm, etc.

The backing 20 may be any suitable structure for supporting the light emitters 22. For example, the backing 20 may be a flexible printed circuit board, and the electrical pathways 26 may be electrical traces located on the flexible processor circuitry.

The light emitters 22 may be positioned across the backing 20. The light emitters 22 may have a specific pattern on the backing 20 such that particular areas of an infant are preferentially illuminated when the phototherapy apparatus 10 is positioned adjacent the infant 12. The light emitters 22 may include any suitable source of light 12 (also referred to as electromagnetic radiation). For example, the light emitters 22 may include light emitting diodes (LEDs), micro-LEDs, organic LEDs (OLEDs), etc.

The light emitters 22 may emit any suitable wavelength of light 12. For example, the light emitters 22 may emit blue light (e.g., 430 nm-520 nm).

The isolating covering 24 may be any suitable structure (e.g., a coating, covering, film, etc.) suitable for electrically isolating the electrical pathways 26 from the external environment 28 (e.g., electrically isolating the electrical pathways 26 and light emitters 22 from an infant 14 placed in contact with the phototherapy apparatus 10. For example, the isolating covering 24 may be a conformal coating applied to the backing 20.

The phototherapy apparatus 10 may additionally include a thermal conductive layer 37. For example, the light source sheet 16 may include a light emitting surface 38 and a back surface 39 opposite the light emitting surface 38. The thermal conductive layer 37 may be thermally connected to the back surface 39, such that the thermal conductive layer 37 draws thermal energy away from the light source sheet 16.

The thermal conductive layer 37 may be any suitable structure for removing heat from the light source sheet 16. For example, the thermal conductive layer 37 may utilize one or more of passive cooling (e.g., a passive heat sink) or active cooling (e.g., thermoelectric cooling). As an example, the thermal conductive layer 37 may be a heat sink (e.g., a metallic sheet) affixed (e.g., using thermal paste) to the back of the light source sheet 16.

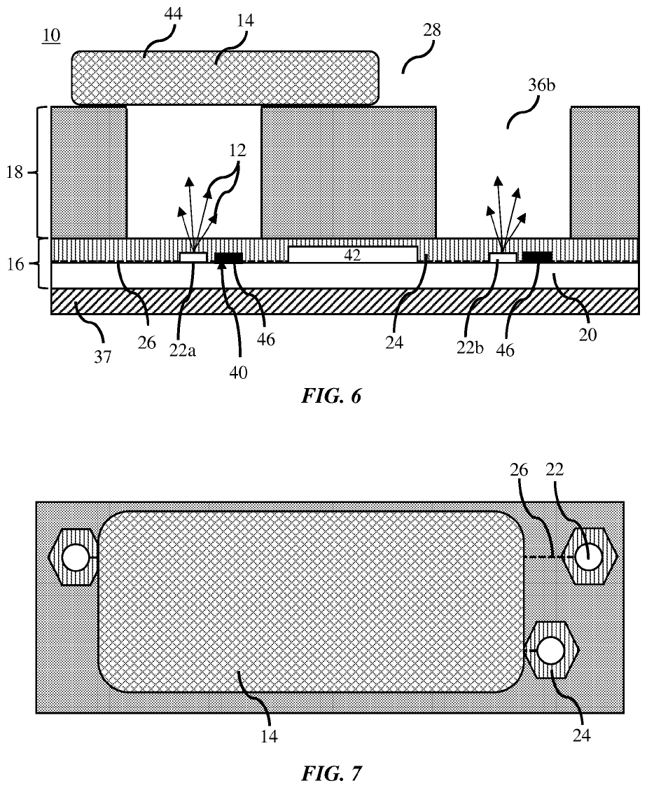

In the embodiment shown in FIGS. 6 and 7, the phototherapy apparatus 10 additionally includes a safety sensor 40 and processor circuitry 42. The safety sensor 40 and processor circuitry 42 may be used to reduce light leakage from the light emitters 22 into the external environment 28. Because the light 12 emitted by the light emitters 22 may include wavelengths that present eye safety issues (e.g., ultraviolet, near ultraviolet, or blue light), individuals near the phototherapy apparatus 10 and the infant 14 receiving the light 12 may be required to wear safety glasses. By reducing light leakage from the phototherapy apparatus 10, it may be possible to avoid the use of such safety glasses.

The processor circuitry 42 may be electrically connected to the light emitters 22 and may be configured to control properties of the light 12 emitted by the light emitters 22. The processor circuitry 42 may modulate the amount of light delivered to the infant via the light emitters 22. For example, the processor circuitry 42 may control at least one of a duration, pattern, wavelength, or intensity of light emitted by the light emitters 22 or portion of light emitters 22. The processor circuitry 42 may modulate the amount of light to provide a therapeutically effective dose to treat jaundice in the infant.

The processor circuitry 42 may have various implementations. For example, the processor circuitry 42 may include any suitable device, such as a processor (e.g., CPU), programmable circuit, integrated circuit, memory and I/O circuits, an application specific integrated circuit, microcontroller, complex programmable logic device, other programmable circuits, or the like. The processor circuitry 42 may also include a non-transitory computer readable medium, such as random-access memory (RAM), a read-only memory (ROM), an erasable programmable read-only memory (EPROM or Flash memory), or any other suitable medium.

The safety sensor 40 identifies covered light emitters 22 that are blocked by an obstruction 44 (e.g., the infant 14), such that the emitted light 12 is blocked by the obstruction 44 from reaching the external environment 28. That is, the safety sensor 40 identifies light emitters 22 that are blocked and that will not leak light 12 into the external environment 28. The processor circuitry 42 controls properties of the light 12 emitted by the light emitters 22, by identifying uncovered light emitters 22 (e.g., those light emitters 22 not identified as covered light emitters 22 by the safety sensor 40) and controls the light emitters 22, such that an output power of the uncovered light emitters 22 is reduced compared to an output power of the covered light emitters. For example, the light emitters 22 identified as uncovered may be turned off, such that the uncovered light sources 22 do not emit light 12.

The safety sensor 40 may include multiple detectors 46. For example, each light emitter 22 may be associated with a detector 46 positioned to detect whether the light emitter 22 is obstructed. In one example, the detector 46 of each of the light emitters 22 is positioned to detect an interaction of the emitted light 12 with an obstruction 44. That is, when the light emitter 22 is obstructed, the detector 46 detects interaction of the light 12 with the obstruction 44 and the light emitter 22 is identified as obstructed. Similarly, when the light emitter 22 is unobstructed, the detector 46 will not detect the interaction of the light 12 with the obstruction 44 and the light emitters 22 may be identified as unobstructed.

The safety sensor 40 may include any suitable sensor for detecting an object. For example, the safety sensor 40 may include one or more force sensor(s) and/or touch sensor(s) (e.g., capacitive touch sensor).

For example, in FIG. 6, the light 12 emitted by light emitter 22a interacts with the infant 14 and a portion of the light 12 is scattered or reflected by the infant 14. This reflected/scattered light is detected by the detector 46a. Conversely, the light 12 emitted by the light emitter 22b does not interact with an obstruction 44 and the light 12 exits the void 36b. For this reason, detector 46b does not detect the light 12 emitted by the light emitter 22b. The detectors 46 may be a photodetector or any suitable device configured to detect light.

In another embodiment, the detectors 46 may detect ambient light (e.g., wavelengths of light not emitted by the light emitters 22). If the detector 46 detects ambient light, then the associated light emitters 22 may be identified as an unobstructed light emitter. Conversely, if the detector 46 does not detect ambient light, then the associated light emitters 22 may be identified as an obstructed light emitter. For example, the detectors 46 may detect wavelengths of red or green light as the ambient light, because these wavelengths may not be emitted by the light emitter 22.

In another embodiment, the detectors 46 may be an object sensor (e.g., a distance sensor) configured to detect obstructions 44 within a specific distance from the detector 46. For example, the detector 46 may detect obstructions 44 located at a distance of the distance of the detector 46 from the external surface 32. The detector 46 may be positioned (e.g., as shown in FIG. 6), such that a detector 46 does not detect an obstruction 44 if the light emitter 22 is unobstructed. If an obstruction 44 is not detected by the detector 46, then the light emitter 22 may be identified as unobstructed. Conversely, if a light emitter 22 is obstructed, then the detector 46 will detect the obstruction 44 and the light emitter 22 may be identified as obstructed.

As described above, the sensor 40 and detectors 46 may be any suitable device for detecting an obstruction 44. For example, the sensor 40 and detectors 46 may be a photodetector, a distance sensor (e.g., an ultrasound sensor), or any suitable device.

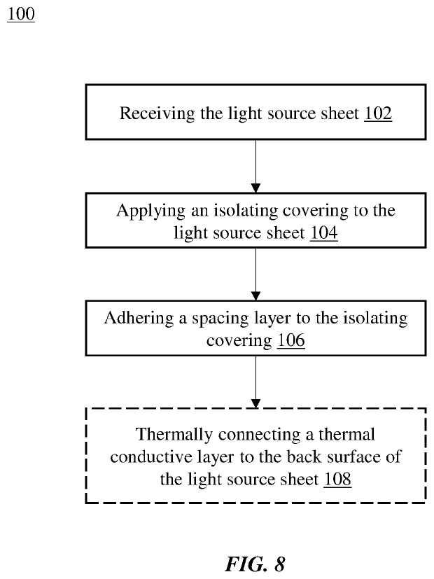

Turning to FIG. 8, an exemplary method 100 is shown for manufacturing a phototherapy apparatus 10 for delivering light 12 to an infant 14. In step 102, the light source sheet 16 is received. As described above, the light source sheet 16 includes a backing 20, light emitters 22 mechanically supported by the backing 20, and electrical pathways 26 located on the backing 20 for delivering electrical power to the light emitters 22. In step 104, the isolating covering 24 is applied to the light source sheet 16, such that the electrical pathways 26 and the light emitters 22 are electrically isolated from the external environment 28. For example, the isolating covering 24 may be applied as a conformal coating 24 to the backing 20.

In step 106, the isolating covering 24 is adhered to the spacing layer 18. The spacing layering 18 is adhered to the isolated covering 24 such that the voids 36 overlap with the light emitters 22 and the light 12 emitted by the light emitters 22 diffuses within the voids 36 before being emitted from the spacing layer 18. For example, the spacing layer 18 may be overmolded onto the isolating covering 24, such that the voids are formed in the spacing layer 18 during the overmolding.

In optional step 108, the thermal conductive layer 37 is thermally connecting to the back surface 39 of the light source sheet 16, such that the thermal conductive layer 37 draws thermal energy away from the light source sheet 16. That is, the light emitters 22 of the light source sheet 22 may generate heat when emitting light 12. This heat may be removed from the light source sheet by the thermal conductive layer 37.

The phototherapy apparatus 10 may additionally include a power source. The power source (e.g., a battery or a plug configured to receive power from an external power source) may be integrated into or attached to the phototherapy apparatus 10 (e.g., mechanically supported by the backing 20).

Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A phototherapy apparatus for delivering light to an infant, the device comprising:
a light source sheet including a backing, light emitters mechanically supported by the backing, an isolating covering, and electrical pathways located on the backing for delivering electrical power to the light emitters, wherein the light emitters are configured to emit light and the isolating covering is configured to electrically isolate the electrical pathways and the light emitters from an external environment; and
a spacing layer configured to diffuse the light from the light emitters before the light is emitted from the spacing layer, wherein the spacing layer contains at least one internal void which is positioned to overlap with at least one light emitter;
at least one sensor located adjacent to the at least one light emitter, wherein the at least one internal void that overlaps the at least one light emitter also overlaps the at least one sensor,
wherein the sensor is configured to identify covered light emitters that are blocked by an obstruction, such that the light emitted by the light emitter is blocked by the obstruction from escaping into an external environment; and
processor circuitry configured to control properties of the light emitted by the light emitters by:
receiving from the sensor the identified covered light emitters;
identifying uncovered light emitters as the light emitters that are not the identified covered light emitters; and
controlling the determined uncovered light emitters, such that an output power of the uncovered light emitters is reduced compared to an output power of the covered light emitters.

2. The phototherapy apparatus of claim 1, wherein:
the spacing layer includes an internal surface and an external surface opposite the internal surface;
the spacing layer is made of a flexible material;
the spacing layer contains a plurality of voids;
the voids are positioned to overlap with the light emitters, such that the light emitted by the light emitters diffuses within the voids; and
a void is associated with a light emitter when the void overlaps the light emitter.

3. The phototherapy apparatus of claim 2, wherein:
the internal surface is mechanically supported by the light source sheet; and the voids extend between the internal surface and the external surface.

4. The phototherapy apparatus of claim 3, wherein the voids comprise hexagonal cutouts.

5. The phototherapy apparatus of claim 1, wherein the spacing layer further includes lensing features positioned to overlap with the light emitters, such that the light emitted by the light emitters is received by the lensing features and upon being emitted from the lensing features is at least one of spread more uniformly or has a reduced angular dispersion.

6. (canceled)

7. The phototherapy apparatus of claim 1, wherein:
the sensor includes multiple detectors;
each light emitter includes an associated detector of the multiple detectors positioned to detect whether the light emitter is obstructed.

8. The phototherapy apparatus of claim 7, wherein, for each light emitter, the associated detector is positioned to detect an interaction of the light emitted by the light emitter with an obstruction of the light emitter when the obstruction is present, such that the light emitter is identified as an uncovered light emitter when the interaction of the emitted light is not detected by the associated detector.

9. The phototherapy apparatus of claim 7, wherein:
the detector is configured to detect ambient light having a different wavelength than a wavelength of the emitted light;
for each light emitter, when the associated detector detects ambient light, identifying the light emitter as an uncovered light emitter; and
for each light emitter, when the associated detector does not detect ambient light, identifying the light emitter as a covered light emitter.

10. The phototherapy apparatus of claim 1, wherein the spacing layer is adhered to the light source sheet.

11. The phototherapy apparatus of claim 10, wherein the spacing layer is adhered to the light source sheet by overmolding the spacing layer onto the light source sheet.

12. The phototherapy apparatus of claim 1, wherein the backing is a flexible printed circuit board, and the electrical pathways are electrical traces located on the flexible processor circuitry.

13. The phototherapy apparatus of claim 1, wherein the isolating covering is a conformal coating applied to the backing.

14. The phototherapy apparatus of claim 1, further comprising a thermal conductive layer, wherein:
the light source sheet includes a light emitting surface and a back surface opposite the light emitting surface; and
the thermal conductive layer is thermally connected to the back surface, such that the thermal conductive layer draws thermal energy away from the light source sheet.

15. The phototherapy apparatus of claim 1, wherein the spacing layer has a thickness of at least 5 mm.

16. A method of manufacturing a phototherapy apparatus for delivering light to an infant, the method comprising:
receiving a light source sheet including a backing, light emitters mechanically supported by the backing, and electrical pathways located on the backing for delivering electrical power to the light emitters, wherein the light emitters are configured to emit light;
applying an isolating covering to the light source sheet, such that the electrical pathways and the light emitters are electrically isolated from an external environment; and
adhering to the isolating covering a spacing layer configured to diffuse the light emitted from the light emitters, wherein the spacing layer includes voids and the spacing layering is adhered to the isolated covering such that the voids overlap with the light emitters and the light emitted by the light emitters diffuses within the voids before being emitted from the spacing layer.

17. The method of claim 16, wherein the adhering of the spacing layer to the isolating covering includes:
overmolding the spacing layer onto the isolating covering, such that the voids are formed in the spacing layer during the overmolding.

18. The method of claim 16, wherein the light source sheet includes a light emitting surface and a back surface opposite the light emitting surface, the method further comprising:
thermally connecting a thermal conductive layer to the back surface of the light source sheet, such that the thermal conductive layer draws thermal energy away from the light source sheet.

19. The method of claim 16, wherein the backing is a flexible printed circuit board, and the electrical pathways are electrical traces located on the flexible processor circuitry.

20. The method of claim 19, applying an isolating covering includes applying the isolating covering as a conformal coating to the backing.
